(19) United States
(12) Patent Application Publication
(10) Pub. No.: US 2024/0164885 A1
(43) Pub. Date: May 23, 2024

(54) BODILY IMPLANTS WITH COATINGS AND METHODS OF MAKING THE SAME

(71) Applicant: Boston Scientific Scimed, Inc., Maple Grove, MN (US)

(72) Inventors: Yen-Hao Hsu, Shrewsbury, MA (US); Heather Cirka, Hudson, MA (US); Niraj Prasad Rauniyar, Plymouth, MN (US)

(21) Appl. No.: 18/509,987

(22) Filed: Nov. 15, 2023

Related U.S. Application Data
(60) Provisional application No. 63/384,653, filed on Nov. 22, 2022.

(51) Int. Cl. A61F 2/00; A61L 31/04; A61L 31/14

(52) U.S. Cl. CPC A61F 2/0063; A61L 31/048; A61L 31/148; A61F 2210/0004; A61L 2420/02

(57) ABSTRACT

An implant includes a substrate having an outer surface, a first layer disposed on the outer surface of the substrate, and a second layer disposed on the outer surface of the substrate. The first layer includes a polymeric material.

### CROSS-REFERENCE TO RELATED APPLICATION

This application claims priority to U.S. Provisional Patent Application No. 63/384,653, filed on Nov. 22, 2022, entitled “BODILY IMPLANTS WITH COATINGS AND METHODS OF MAKING THE SAME”, the disclosure of which is incorporated by reference herein in its entirety.

### TECHNICAL FIELD

This disclosure relates generally to bodily implants with coatings and methods of making bodily implants with coatings.

### BACKGROUND

Bodily implants may be placed within a body for a variety of reasons. For example, bodily implants, such as mesh implants, may be placed within a body of a patient to provide or help provide support to a portion of the body of the patient. Such bodily implants may be sized or shaped to achieve the proposed function within the body of the patient.

### SUMMARY

According to an implementation, an implant includes a substrate having an outer surface, a first layer disposed on the outer surface of the substrate, and a second layer disposed on the outer surface of the substrate. The first layer includes a polymeric material.

In some implementations, the substrate includes a mesh material. In some implementations, the substrate includes a polypropylene mesh material. In some implementations, the substrate defines openings. In some implementations, the substrate includes a hydrocarbon polymer. In some implementations, the substrate includes a member having a substantially solid outer surface.

In some implementations, the first layer is formed of a bioresorbable material. In some implementations, the first layer is formed of a material that is non-bioresorbable.

In some implementations, the second layer is a hydrophobic layer. In some implementations, the second layer is a hydrophilic layer.

In some implementations, the outer surface is a first outer surface, the substrate having a second outer surface. In some implementations, the outer surface is a first outer surface, the substrate having a second outer surface, the second outer surface being spaced from the first outer surface, and the implant further includes, a third layer disposed on the second outer surface of the substrate, the first layer including a polymeric material, and a fourth layer disposed on the outer surface of the substrate, the third layer being disposed between the fourth layer and the second outer surface of the substrate.

In some implementations, the outer surface is a first outer surface, the substrate having a second outer surface, the second outer surface being disposed opposite the first outer surface, and the implant further includes, a third layer disposed on the second outer surface of the substrate, the first layer including a polymeric material, and a fourth layer disposed on the outer surface of the substrate, the third layer being disposed between the fourth layer and the second outer surface of the substrate.

In some implementations, the substrate includes a mesh material, the outer surface is a first outer surface, the substrate having a second outer surface, the second outer surface being disposed opposite the first outer surface, the implant further including, a third layer disposed on the second outer surface of the substrate, the first layer including a polymeric material, and a fourth layer disposed on the outer surface of the substrate, the third layer being disposed between the fourth layer and the second outer surface of the substrate.

In some implementations, the substrate includes a first portion, a second portion, and a third portion, the first portion, the second portion, and the third portion forming a Y-shaped substrate.

According to another implementation, a method of forming an implant includes treating an outer surface of a substrate, applying a first layer of material to the outer surface of the substrate, and applying a second layer of material to the outer surface of the substrate.

In some implementations, the treating the outer surface of the substrate includes a plasma treatment process.

In some implementations, the applying a first layer of material to the outer surface of the substrate includes applying a bioabsorbable layer to the outer surface of the substrate.

In some implementations, the applying a second layer of material to the outer surface of the substrate includes applying a hydrophobic material to the outer surface of the substrate. In some implementations, the applying a second layer of material to the outer surface of the substrate includes applying a hydrophilic material to the outer surface of the substrate.

In some implementations, the substrate includes a mesh material.

### BRIEF DESCRIPTION OF THE DRAWINGS

FIG. 1 schematically illustrates an implant according to an implementation.

FIG. 2 schematically illustrates an implant according to an implementation.

FIG. 3 is a top view of an implant according to an implementation.

FIG. 4 schematically illustrates the implant of FIG. 3 disposed within a body of a patient.

FIG. 5 is a perspective view of an implant according to an implementation.

FIG. 6 schematically illustrates the implant of FIG. 5 disposed within a body of a patient.

FIG. 7 illustrates a surface treatment process.

FIG. 8 illustrates a process to dispose a first layer of material on a surface of an implant.

FIG. 9 illustrates a process to dispose a second layer of material on a surface of an implant.

FIGS. 10-13 illustrate example hydrophilic coatings.

FIGS. 14-16 illustrate example hydrophobic coatings.

FIG. 17 is a flow chart of a method of forming an implant according to an implementation.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. For example, while the specific implementations are directed to implants, such as bodily implants, it should be understood that the processes and methods may be used in other areas or fields as well. For example, in some instances, the processes and methods disclosed herein may be useful for objects or devices in other environmental settings, such as tubes or pipes that are exposed to certain environmental surroundings. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly, mechanically, or chemically.

In general, the implementations are directed to medical devices such as bodily implants or other devices configured to be placed within the body of a patient. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some implementations, the patient may be a human male, a human female, or any other mammal.

The implementations discussed herein may improve the performance of a bodily implant. For example, the bodily implant may include coatings or layers of material that are configured to prevent or help prevent the adherence or absorption of proteins by the bodily implant while the implant is disposed within the body. Accordingly, the lack of protein absorption may also help prevent macrophage adhesion to the bodily implant, encapsulation of the implant, and/or capsular contracture.

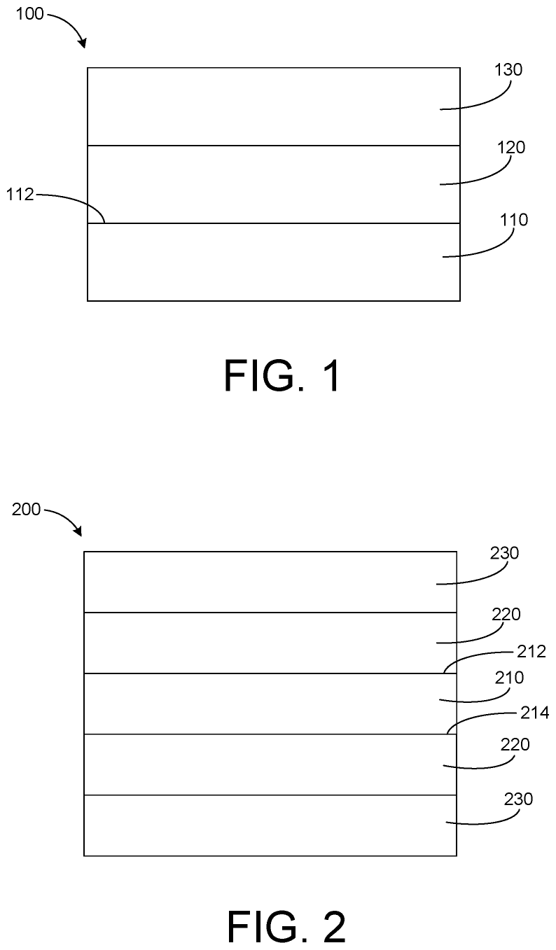

FIG. 1 schematically illustrates a bodily implant 100 according to an implementation. In some implementations, the bodily implant 100 may be a device configured to be placed into the body and remain within the body of the patient. In some implementations, the bodily implant 100 may be a device configured to be placed into the body of the patient and provide support or support structure to the body of the patient. In other implementations, the bodily implant 100 may be a device configured to provide a different function within the body of the patient.

In the illustrated implementation, the implant 100 includes a substrate 110, a first layer of material 120, and a second layer of material 130. The substrate 110 may be of any shape or form. In some implementations, the substrate 110 is a mesh material such as a knit or woven mesh that defines openings. In other implementations, the substrate 110 is a material that does not include or define openings. In yet other implementations, the substrate 110 may be a tubular member or other type of housing that is configured to retain or house components of the medical device. In such implementations, the outer surface may be substantially solid or free of openings.

In some implementations, the substrate 110 is formed of a polypropylene material. For example, the substrate 110 may be a knit or woven mesh formed of polypropylene strands. In other implementations, the substrate 110 is formed of a different polyolefin. In some implementations, the substrate is formed of a different hydrocarbon polymer such as polyethylene, isotactic polypropylene, syndiotactic polypropylene, polyisobutylene, or polybutadiene.

The first layer 120 is formed on or otherwise attached to an outer surface 112 of the substrate 110. In some implementations, the first layer 120 is formed of a polymeric material. Additionally, in some implementations, the first layer 120 is formed of a biodegradable or bioresorbable material. For example, in some implementations, the first layer 120 is formed of a biodegradable polyester. In other implementations, the first layer 120 is formed of a non-biodegradable material or a non-bioresorbable material.

The second layer 130 is formed on or otherwise attached to the outer surface 112 of the substrate 110. In some implementations, the second layer 130 is formed or otherwise attached to the outer surface 112 of the substrate such that the first layer 120 is disposed between the outer surface 112 and the second layer 130. In some implementations, the second layer 130 is formed of a hydrophobic material. In other implementations, the second layer 130 is formed of a hydrophilic material. In some implementations, the second layer 130 is configured to prevent or help prevent the attachment of proteins, such as fibrinogen or other bodily proteins, to the implant 100. In some implementations, the second layer 130 is configured to prevent or help prevent the absorption of proteins, such as fibrinogen or other bodily proteins, by or on the implant 100.

In the illustrated implementation, the first layer 120 and the second layer 130 are formed or attached to one of the outer surfaces of the substrate 110. As illustrated schematically in FIG. 2, in other implementations, the first layer 220 and the second layer 230 are formed on or attached to a second outer surface of the substrate.

FIG. 2 schematically illustrates a bodily implant 200 according to an implementation. In some implementations, the bodily implant 200 may be a device configured to be placed into the body and remain within the body of the patient. In some implementations, the bodily implant 200 may be a device configured to be placed into the body of the patient and provide support or support structure to the body of the patient. In other implementations, the bodily implant 200 may be a device configured to provide a different function within the body of the patient.

In the illustrated implementation, the implant 200 includes a substrate 210 that has a first outer surface 212 and a second outer surface 214. In some implementations, the first outer surface 212 is spaced from or disposed apart from the second outer surface 214. For example, in some implementations, the second outer surface 214 may be disposed on an opposite side of the substrate from the first outer surface 212. In the illustrated implementation, the implant 200 includes a first layer of material 220 and a second layer of material 230. The first layer of material 220 is attached to or disposed on the first outer surface 212 and the second outer surface 214. In other words, the first outer surface 212 has a first layer of material coupled thereto and the second outer surface 214 has a first layer of material coupled thereto. The second layer of material 220 is attached to or disposed on the first outer surface 212 and the second outer surface 214. In other words, the first outer surface 212 has a second layer of material coupled thereto and the second outer surface 214 has a second layer of material coupled thereto.

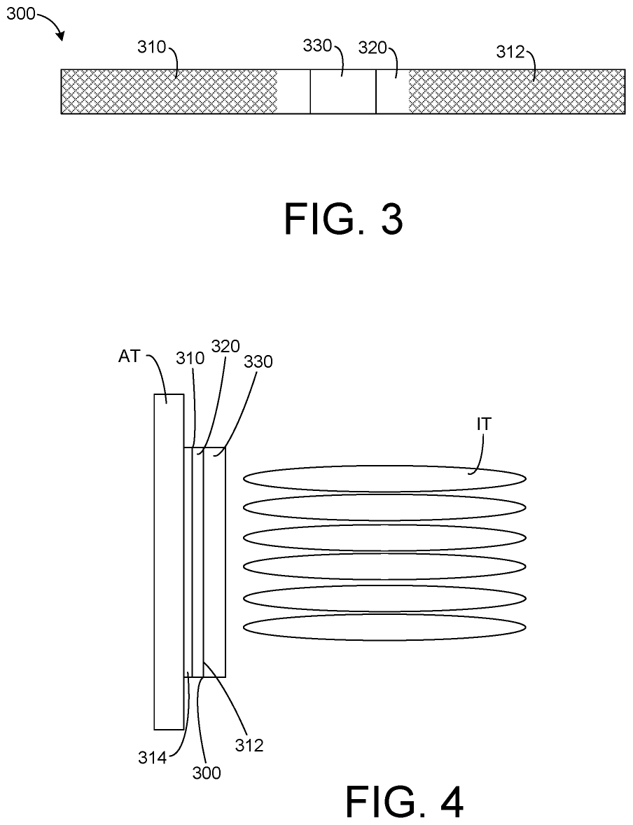

FIG. 3 illustrates a bodily implant 300 according to an implementation. FIG. 4 schematically illustrates the bodily implant 300 disposed within a body of a patient. The implant 300 includes a substrate 310, a first layer of material 320, and a second layer of material 330. The substrate 310 may be of any shape or form. The substrate 310 is a mesh material such as a knit or woven mesh that defines openings.

In the illustrated implementation, the substrate 310 is formed of a polypropylene material. The substrate 310 is a knit or woven mesh formed of polypropylene strands. In other implementations, the substrate 310 is formed of a different polyolefin. In some implementations, the substrate is formed of a different hydrocarbon polymer such as polyethylene, isotactic polypropylene, syndiotactic polypropylene, polyisobutylene, or polybutadiene.

The substrate 310 includes a first outer surface 312 and a second outer surface 314 disposed opposite the first outer surface 312. In other words, the first outer surface 312 is disposed on one side of the substrate 310 and the second outer surface 314 is disposed on an opposite side of the substrate.

The first layer 320 is formed on or otherwise attached to the first outer surface 312 of the substrate 310. In the illustrated implementation, the first layer 320 is only formed on a portion of the first outer surface 312. In other implementations, the first layer 320 is disposed or formed on the entirety of the first outer surface 312. In some implementations, the first layer 320 is formed of a polymeric material. Additionally, in some implementations, the first layer 320 is formed of a biodegradable or bioresorbable material. For example, in some implementations, the first layer 320 is formed of a biodegradable polyester. In other implementations, the first layer 320 is formed of a non-biodegradable material or a non-bioresorbable material.

The second layer 330 is formed on or otherwise attached to the outer surface 312 of the substrate 310. In some implementations, the second layer 330 is formed or otherwise attached to the outer surface 312 of the substrate such that the first layer 320 is disposed between the outer surface 312 and the second layer 330. In the illustrated implementation, the second layer 330 is only formed on a portion of the first outer surface 312. In other implementations, the second layer 330 is disposed or formed on the entirety of the first outer surface 312. In some implementations, the second layer 330 is formed of a hydrophobic material. In other implementations, the second layer 330 is formed of a hydrophilic material. In some implementations, the second layer 330 is configured to prevent or help prevent the attachment of proteins, such as fibrinogen or other bodily proteins, to the implant 300. In some implementations, the second layer 330 is configured to prevent or help prevent the absorption of proteins, such as fibrinogen or other bodily proteins, by the implant 300.

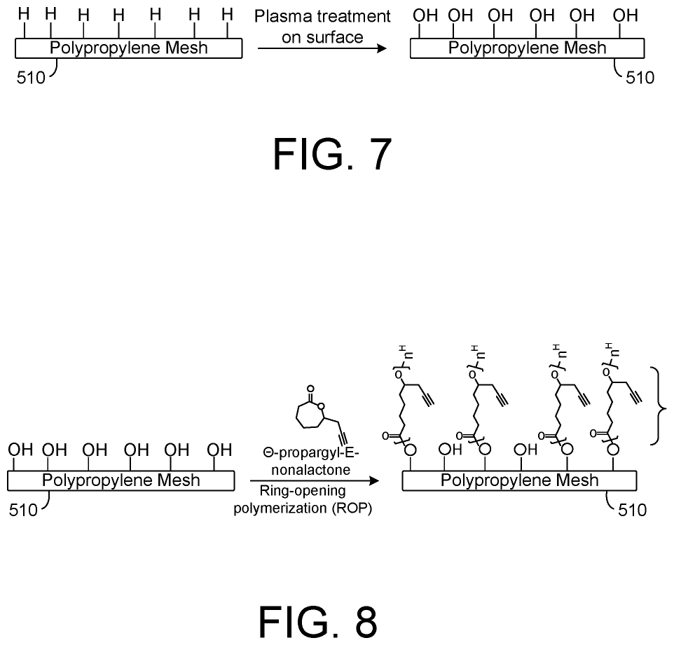

In the illustrated implementation, the second outer surface 314 does not include any layers of material attached thereto. In other words, the second outer surface 314 is devoid of layers of additional material. A process for forming layers of material on the surface of the substrate is discussed in detail below with respect to FIGS. 7-9.

In some implementations, the bodily implant 300 may be a device configured to be placed into the body and remain within the body of the patient. In some implementations, the bodily implant 300 may be a device configured to be placed into the body of the patient and provide support or support structure to the body of the patient. In other implementations, the bodily implant 300 may be a device configured to provide a different function within the body of the patient.

As best illustrated in FIG. 4, in the illustrated implementation, the implant 300 may be placed within the body of a patient adjacent abdominal tissue AT and intestinal tissue IT. The implant 300 may be configured to provide support to the body of the patient, such as to the abdominal tissue AT. In some cases, the implant 300 may be used to treat a hernia. As illustrated, the implant 300 may be placed within the body of the patient such that the first outer surface 312 (the side with the layers of material) faces the intestinal tissue IT and the second outer surface 314 faces the abdominal tissue AT. In some cases, the layers of material are configured to help prevent the attachment of bodily proteins or bodily tissue to the implant 300.

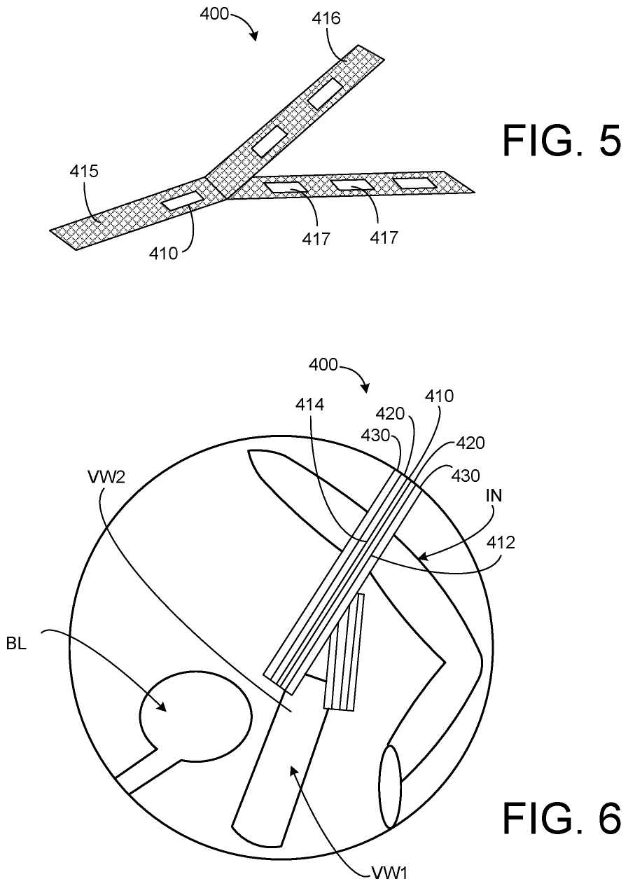

FIG. 5 illustrates a bodily implant 400 according to an implementation. FIG. 6 schematically illustrates the bodily implant 400 disposed within a body of a patient. The implant 400 includes a substrate 410, a first layer of material 420, and a second layer of material 430. The substrate 410 is a mesh material such as a knit or woven mesh that has defined openings.

In the illustrated implementation, the substrate 410 includes a first portion or first leaf 415, a second portion or second leaf 416, and a third portion or third leaf 417. The first leaf 415, second leaf 416, and the third leaf 417 are coupled together at a junction 418 to form a Y-shaped implant. Any known method may be used to couple the leaves 415, 416, and 417 together. For example, in some implementations, the leaves 415, 416, and 417 are stitched together. In some implementations, more than one of the leaves 415, 416, and 417 may be formed unitarily or with a single knitting or weaving process.

In the illustrated implementation, the substrate 410 (including the first leaf 415, the second leaf 416, and the third leaf 417) is formed of a polypropylene material. The substrate 410 is a woven mesh formed of polypropylene strands. In other implementations, the substrate 410 is formed of a different polyolefin. In some implementations, the substrate 410 is formed of a different hydrocarbon polymer such as polyethylene, isotactic polypropylene, syndiotactic polypropylene, polyisobutylene, or polybutadiene.

The substrate 410 includes a first outer surface 412 and a second outer surface 414 disposed opposite the first outer surface 412. In other words, the first outer surface 412 is disposed on one side of the substrate 410 and the second outer surface 414 is disposed on an opposite side of the substrate 410.

The first layer 420 is formed on or otherwise attached to the first outer surface 412 of the substrate 410 and to the second outer surface 414 of the substrate 410. In some implementations, the first layer 420 is formed of a polymeric material. Additionally, in some implementations, the first layer 420 is formed of a biodegradable or bioresorbable material. For example, in some implementations, the first layer 420 is formed of a biodegradable polyester. In other implementations, the first layer 420 is formed of a non-biodegradable material or a non-bioresorbable material.

The second layer 430 is formed on or otherwise attached to the first outer surface 412 of the substrate 410 and to the second outer surface 414 of the substrate 410. In some implementations, the second layer 430 is formed or otherwise attached to the first outer surface 412 of the substrate 410 such that the first layer 420 is disposed between the first outer surface 412 and the second layer 430. Similarly, on the opposite side of the implant 400, the second layer 430 is formed or otherwise attached to the second outer surface 414 of the substrate 410 such that the first layer 420 is disposed between the second outer surface 414 and the second layer 430. In some implementations, the second layer 430 is formed of a hydrophobic material. In other implementations, the second layer 430 is formed of a hydrophilic material. In some implementations, the second layer 430 is configured to prevent or help prevent the attachment of proteins, such as fibrinogen or other bodily proteins, to the implant 400. In some implementations, the second layer 430 is configured to prevent or help prevent the absorption of proteins, such as fibrinogen or other bodily proteins, by the implant 400.

A process for forming layers of material on the surface of the substrate 410 is discussed in detail below with respect to FIGS. 7-9.

In some implementations, the bodily implant 400 may be a device configured to be placed into the body and remain within the body of the patient. In some implementations, the bodily implant 400 may be a device configured to be placed into the body of the patient and provide support or support structure to the body of the patient. In other implementations, the bodily implant 400 may be a device configured to provide a different function within the body of the patient.

As best illustrated in FIG. 6, in the illustrated implementation, the implant 400 may be placed within the body within a pelvic region of a patient. For example, the implant 400 may be placed within the body near the bladder BL and the intestines IN of the patient. In the illustrated implementation, the implant 400 is placed such that two portions of the implant 400 are coupled to vaginal walls VW1 and VW2 of the patient. The third portion of the implant 400 may be coupled to a support location within the body of the patient. The implant 400 may be configured to provide support to the body of the patient, such as to the vaginal walls of the patient. In other implementations, the implant 400 is placed within a different portion of the body of the patient or is placed within the body for a different purpose.

FIGS. 7-9 schematically illustrate a process for forming layers of material on an outer surface of a substrate. This process may be used to form the layers of material on the outer surfaces of the substrates of the above-described implementations.

As illustrated in FIG. 7, the outer surface of the implant or substrate is prepared. In some implementations, hydroxyl groups are formed on an outer surface of the substrate 510. In the illustrated implementation, the hydroxyl groups are formed on the surface of the substrate via a plasma process. The plasma process is used to activate the surface and form the hydroxyl groups on the surface. In other implementations, a different process is used to prepare the surface of the substrate. For example, in other implementations, a different process is used to form hydroxyl groups on the surface of the implant. In the illustrated implementation, the hydroxyl groups are only formed on one of the surfaces of the substrate. In other implementations, hydroxyl groups may be formed on other surfaces of the substrate 510.

As illustrated in FIG. 8, a biodegradable or bioresorbable layer of material may be coupled to the surface of the substrate. In other implementations, a non-biodegradable or non-bioresorbable layer may be formed. In the illustrated implementation, the substrate 510 is exposed to θ-propargyl-ε-nonalactone and a ring opening polymerization process occurs. In some implementations, the substrate 510 is exposed to the θ-propargyl-ε-nonalactone via a soaking or a dipping process. In other implementations, a different process is used to expose the substrate 510 to the θ-propargyl-ε-nonalactone.

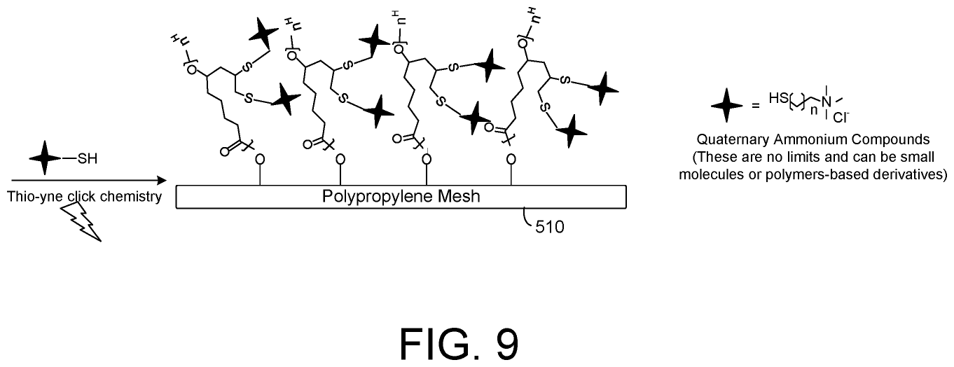

As best illustrated in FIG. 9, a second layer of material may be added to the substrate 510. The second layer of material may be a hydrophobic or a hydrophilic material. In some implementations, the second layer of material may prevent or help prevent the attachment of bodily proteins to the substrate 510. In the illustrated implementation, the substrate 510 is exposed to quaternary ammonium thiol derivatives (illustrated in FIG. 11) and the material is attached to the surface of the substrate via thiol-yne click chemistry. The ammonium thiol derivatives may be small molecules or may be polymer based. In other words, the material is joined with the layer or material that was added to the surface in the process shown in FIG. 8. In some implementations, the quaternary ammonium thiol derivatives are exposed to the substrate via a soaking or a dipping process. In other implementations, a different process is used to expose the substrate 510 to the quaternary ammonium thiol derivatives.

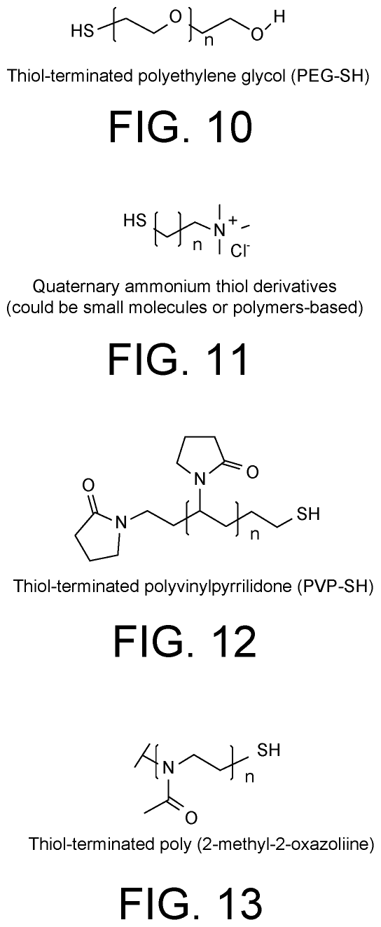

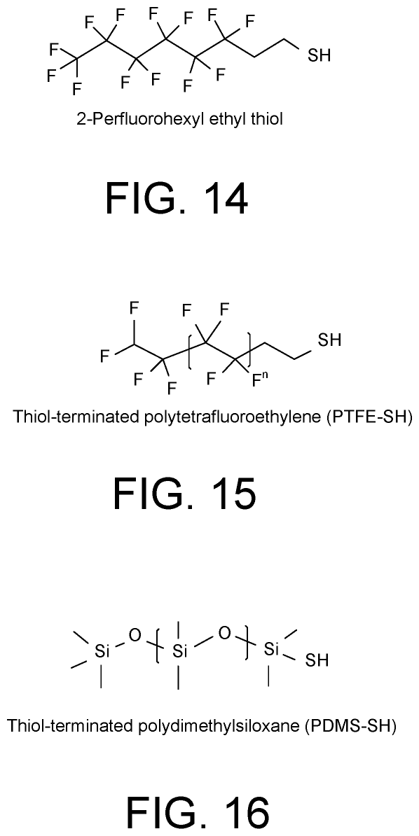

While FIG. 9 illustrates the substrate being exposed to quaternary ammonium thiol derivatives, in other implementations, other compounds are used. For example, as illustrated in FIGS. 10-13, hydrophobic layers may be formed using thiol-terminated polyethylene glycol, quaternary ammonium thiol derivatives, thiol-terminated polyvinylpyrrolidone, or thiol-terminated poly (2-methyl-2-oxazoline). FIGS. 14-16 illustrate compounds that may be used to form a hydrophobic layer, including 2-perflourohexyl thiol, thiol-terminated polytetrafluoroethylene, and thiol-terminated polydimethylsiloxane.

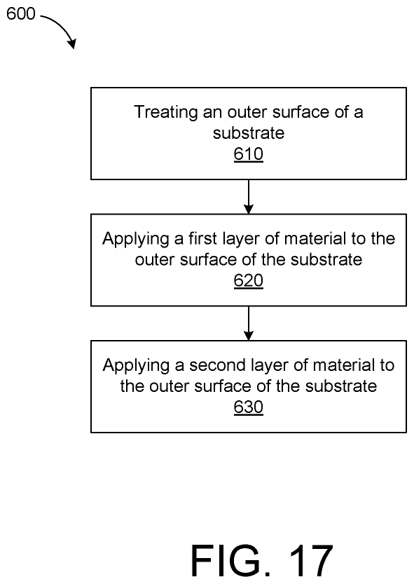

FIG. 17 illustrates a flow chart for a method 600 of forming layers of material on a substrate such as a medical device. At 610, the outer surface or surfaces are treated. This may be done in a manner as described above with respect to FIG. 7. Specifically, hydroxyl groups are formed on a surface of the substrate via a plasma process. At 620, a first layer of material is applied to the outer surface or outer surfaces of the substrate. This may be done in a manner as described above with respect to FIG. 8. At 630, a second layer of material is applied to the outer surface or outer surfaces of the substrate. This may be done in a manner as described above with respect to FIG. 9.

While certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the implementations.

What is claimed is:

1. An implant, comprising:
a substrate having an outer surface;
a first layer disposed on the outer surface of the substrate, the first layer including a polymeric material; and
a second layer disposed on the outer surface of the substrate.

2. The implant of claim 1, wherein the substrate includes a mesh material.

3. The implant of claim 1, wherein the substrate includes a polypropylene mesh material.

4. The implant of claim 1, wherein the substrate defines openings.

5. The implant of claim 1, wherein the substrate includes a hydrocarbon polymer.

6. The implant of claim 1, wherein the substrate includes a member having a substantially solid outer surface.

7. The implant of claim 1, wherein the first layer is formed of a bioresorbable material.

8. The implant of claim 1, wherein the first layer is formed of a material that is non-bioresorbable.

9. The implant of claim 1, wherein the second layer is a hydrophobic layer.

10. The implant of claim 1, wherein the second layer is a hydrophilic layer.

11. The implant of claim 1, wherein the outer surface is a first outer surface, the substrate having a second outer surface.

12. The implant of claim 1, wherein the outer surface is a first outer surface, the substrate having a second outer surface, the second outer surface being spaced from the first outer surface, further comprising,
a third layer disposed on the second outer surface of the substrate, the first layer including a polymeric material; and
a fourth layer disposed on the outer surface of the substrate, the third layer being disposed between the fourth layer and the second outer surface of the substrate.

13. The implant of claim 1, wherein the outer surface is a first outer surface, the substrate having a second outer surface, the second outer surface being disposed opposite the first outer surface, further comprising,
a third layer disposed on the second outer surface of the substrate, the first layer including a polymeric material; and
a fourth layer disposed on the outer surface of the substrate, the third layer being disposed between the fourth layer and the second outer surface of the substrate.

14. The implant of claim 1, wherein substrate includes a mesh material, the outer surface is a first outer surface, the substrate having a second outer surface, the second outer surface being disposed opposite the first outer surface, further comprising,
a third layer disposed on the second outer surface of the substrate, the first layer including a polymeric material; and
a fourth layer disposed on the outer surface of the substrate, the third layer being disposed between the fourth layer and the second outer surface of the substrate.

15. The implant of claim 1, wherein the substrate includes a first portion, a second portion, and a third portion, the first portion, the second portion, and the third portion forming a Y-shaped substrate.

16. A method, comprising:
treating an outer surface of a substrate;
applying a first layer of material to the outer surface of the substrate; and
applying a second layer of material to the outer surface of the substrate.

17. The method of claim 16, wherein the treating the outer surface of the substrate includes a plasma treatment process.

18. The method of claim 16, wherein the applying a first layer of material to the outer surface of the substrate includes applying a bioabsorbable layer to the outer surface of the substrate.

19. The method of claim 16, wherein the applying a second layer of material to the outer surface of the substrate includes applying a hydrophobic material to the outer surface of the substrate.

20. The method of claim 16, wherein the substrate includes a mesh material.
